(19) United States
(12) Patent Application Publication
(10) Pub. No.: US 2023/0053816 A1
(43) Pub. Date: Feb. 23, 2023

(54) Egg Characteristic Determining Device

(71) Applicant: In Ovo Holding B.V., Leiden (NL)

(72) Inventors: Wil Marijn Stutterheim, Leiden (NL); Leonard Van Bommel, Leiden (NL); Wouter Sebastiaan Bruins, Leiden (NL); Edwin Dijksterhuis, Enschede (NL); Jarno Van De Wel, Enschede (NL); Michiel Leemker, Enschede (NL)

(73) Assignee: In Ovo Holding B.V., Leiden (NL)

(21) Appl. No.: 17/758,702

(22) PCT Filed: Jan. 18, 2021
(86) PCT No.: PCT/NL2021/050028

§ 371 Date: Jul. 13, 2022

(30) Foreign Application Priority Data
Jan. 17, 2020 (NL) 2024699

(51) Int. Cl. A01K 43/00; G01N 33/08

(52) U.S. Cl. CPC A01K 43/00; G01N 33/085

(57) ABSTRACT

The present invention relates to an automated egg analyzing and determining system, comprising: a conveyor system configured to transport one or more eggs to a sampling system; a sampling system configured to extract a sample from one or more eggs; a sample transfer system configured to receive the sample for transfer to an assaying system; and an assaying system configured to receive the sample from the sample transfer system and to determine one or more characteristics of one or more eggs or the sample or an aliquot of a sample thereof.

### FIELD OF THE INVENTION

The present application provides a system for taking and analysing samples from eggs, for transferring these samples to an assaying system and determining one or more characteristics of the eggs.

### BACKGROUND OF THE INVENTION

Discrimination between poultry eggs, hereinafter “eggs”, on the basis of some observable quality is a well-known and long-used practice in the poultry industry. “Candling” is a common name for one such technique, a term which has its roots in the original practice of inspecting an egg using the light from a candle. Although eggshells appear opaque under most lighting conditions, eggs are actually somewhat translucent. Accordingly, when placed in front of a light source, the contents of an egg can be observed.

In poultry hatcheries, one purpose of candling eggs is to identify and then segregate live eggs, i.e., eggs which are to be hatched to live poultry, from non-live eggs, e.g., clear eggs, dead eggs, rotted eggs, empty eggs, unfertilized eggs, and the like. U.S. Pat. Nos. 4,955,728 and 4,914,672, both to Hebrank, describe a candling apparatus that uses infrared detectors and the infrared radiation emitted from an egg to identify live eggs. U.S. Pat. No. 4,671,652 to van Asselt et al. describes a candling apparatus in which a plurality of light sources and corresponding light detectors are mounted in an array, and wherein eggs are passed between the light sources and the light detectors to identify live eggs.

In hatchery management, it may be desirable to separate birds based upon various characteristics, such as sex, diseases, genetic traits, etc. For example, it may be desirable to inoculate male birds with a particular vaccine and inoculate female birds with a different vaccine. Sex separation of birds at hatch may be important for other reasons as well. For example, turkeys are conventionally segregated by sex because of the difference in growth rate and nutritional requirements of male and female turkeys. In the layer or table egg industry, it is desirable to keep only females. In the broiler industry, it is desirable to segregate birds based on sex to gain feed efficiencies, improve processing uniformity, and reduce production costs.

Unfortunately, conventional methods of sexing birds may be expensive, labour intensive, time consuming, and typically require trained persons with specialized skills. Conventional methods of sexing birds include feather sexing, vent sexing, and DNA or blood sexing. About three thousand (3,000) chicks can be feather-sexed per hour at a cost of about 0.7 to 2.5 cents per chick. About fifteen hundred (1,500) chicks can be vent-sexed per hour at a cost of about 3.6 to 4.8 cents per chick. DNA or blood sexing is performed by analyzing a small sample of blood collected from a bird. Also, usually male birds are usually terminated directly after hatching of they have no other use. Accordingly, hitherto known methods of determining the gender of birds in ovo are considered too slow, too impractical and too unreliable for industrializing purposes.

It would be desirable to identify the sex of birds, as well as other characteristics of birds, prior to hatching. Pre-hatch sex identification could reduce costs significantly for various members of the poultry industry. Although conventional candling techniques can discriminate somewhat effectively between live and non-live eggs, these conventional candling techniques may not be able to reliably determine sex and other characteristics of unhatched birds.

More advanced methods and devices for pre-hatch sex identification and subsequent sorting are described in. U.S. Pat. No. 8,610,018B2 and WO2015179719A1. These disclose a system comprising an egg conveyer belt, a sampling system extracting material from an egg, an assaying system and a sorting system based on a characteristic identified by the assaying system. These systems however describe the deposit of the sample material on a tray or template followed by external transport from the sampling system to the assaying system and fail to provide a means for a direct in-line connection from the sampling system to the assaying system, which would preserve sample integrity and give additional options for sample preparation prior to analysis.

Mass spectrometry is a powerful analyte detection and measurement technique that has become the preferred method of detecting small molecule, amino acid, protein, peptide, nucleic acid, lipid, and carbohydrate analytes to a high accuracy for diagnostic purposes. Some specimens have a limited shelf-life due to deterioration of one or more analytes or evaporation which distorts the concentration of the analyte. Mass spectrometry, is a technique that can be easily used for online direct analyses, thereby preventing long shelf-life and hence, deterioration.

WO2005009202A2 describes a method and system for automatic identification of bioagents via robotic DNA extraction, PCR and ultimately mass spectrometry, but does not provide a means for taking and directly transferring samples from eggs. EP3185016A1 describes a system layout for an automated system for sample preparation and analysis by mass spectrometry, but also does not provide a means for taking and directly transferring samples from eggs.

WO2017204636A discloses a method and system for identifying a characteristic of an egg comprising obtaining a sample. preferably from the allantois of the egg, and analyzing the sample by mass spectrometry on certain biomarkers.

None of the above prior art discloses a candling unit wherein a light source is positioned between the egg and a detector.

Hence there remains a need for an automated device that provides a means for taking samples from eggs that can transfer the samples for MS analysis in a speedy and direct manner and subsequently sort the sampled eggs based on the results of the analysis.

### SUMMARY OF THE INVENTION

In view of the above discussion, aspects of the present disclosure provide methods of processing eggs having an identified characteristic, e.g. sex, wherein material, e.g. allantoic fluid, is extracted from each of a plurality of eggs, the extracted material is assayed to identify eggs having a characteristic, and then eggs identified as having the characteristic are processed accordingly. For example, a method of processing eggs based upon sex, according to aspects of the present disclosure, includes extracting material from the eggs, assaying each live egg to identify the sex of each live egg. According to aspects of the present disclosure, an automated egg determining system is provided and includes four independent modules linked via a network.

The object of present invention is therefore to provide a system for determining eggs, comprising:
a. a conveyor system configured to transport one or more eggs to a sampling system;
b. a sampling system configured to extract a sample from one or more eggs;
c. a sample transfer system configured to receive the sample for transfer to an assaying system; and
d. an assaying system configured to receive the sample from the sample transfer system and to determine one or more characteristics of one or more eggs or the sample or an aliquot of a sample thereof.

The sampling system advantageously comprises a candling unit comprising one or more light sources and one or more detectors, wherein each of the at least one detector is positioned relative to the light source such that the detector can detect an image of the egg via light originating from the light source. This light source is advantageously positioned between the egg and the detector.

It is a further object to provide a system that comprises a sorting system in communication with the assaying system and configured to sort the egg based on the characteristics of the egg as determined by the assaying system.

It is yet a further object to provide a system wherein the sampling system comprises a candling unit which comprises one or more light sources and one or more detectors.

In a further aspect, the subject system comprises a candling unit comprising a spacer system comprising a spacer object.

In yet a further aspect, the subject system comprises a system that is configured to, and operable to position the egg to be in lateral contact with one or more, preferably three or four, movable objects configured to move into or out of contact with the egg to respectively hold the egg or release the egg, preferably prior to, or during candling.

It is a further object to provide a system that comprises movable objects comprising an elastic object that stores mechanical energy.

It is yet a further object to provide a system comprising a sampling system comprising one or more openers to open a part of the egg shell of one or more eggs. An “opener” herein refers to any opening device capable of piercing or otherwise penetrating the outer calcified shell of an egg for introduction of an extractor. Examples include, but are not limited to punches, drills, or otherwise mechanical opening devices.

In a further aspect, the subject system comprises a sampling system comprising one or more extractors to extract a sample from one or more eggs.

The present invention also relates to a process of determining eggs comprising a system according to any of the preceding systems of the present invention.

In addition, the present invention relates to the process of determining eggs comprising the steps of:
a. candling one or more eggs;
b. automatically extracting one or more samples from one or more eggs;
c. transferring the sample to be analyzed, to an analyzing unit;
d. automatically analyzing the sample or an aliquot of a sample thereof, and
e. determining one or more characteristics of the egg.

Finally, the present invention relates to the use of a system for automated determination of a characteristic in a multitude of eggs according to any of the preceding systems of the present invention.

### SHORT DESCRIPTION OF THE FIGURES

FIG. 1 shows a flow chart for a system for taking samples from eggs, transferring the samples to an assaying system and determining one or more characteristics of the eggs.

FIG. 2 shows a model of a device capable of holding one or more eggs, which is also capable of rotating the egg and/or positioning the egg relative to a candling unit.

FIG. 3 shows a schematic overview of different steps of a system for taking one or more samples from one or more eggs.

FIG. 4 shows a detailed schematic cross section of an extractor positioned in a (chicken) egg at the moment before taking a sample.

FIG. 5 shows a schematic cross section of a hollow elongated object of an extractor positioned in an egg.

FIG. 6 shows a schematic overview of the egg and a spacer system during candling.

FIG. 7 shows a schematic of the egg in contact with a spacer object and out of contact with flat springs prior to being rotated.

### DETAILED DESCRIPTION OF THE INVENTION

The present invention relates to an automated egg determining system. The term “egg determining system” refers to a system adapted for, and configured to determine one or more characteristics of an egg, such as sex of the embryo; development and health of the embryo, and other characteristics such as whether the embryo is still alive or recently deceased. For incubated chicken eggs, the length of the first incubating period is between 6 to 13 days. It would be desirable to determine the sex of an embryo in the egg as early as possible, for instance by the presence and amount of a gender specific compound such that it may be reliably detected. At the same time, it would be desirable that the embryo is still at a stage such before the nervous system of the embryo has reached a stage in which the embryo is able to perceive pain.

The term “automated” refers to system that operates autonomically, i.e. without user intervention, other than e.g. placing the one or more, preferably a multitude of eggs into the system, and removing eggs after sampling.

Candling is a common method used in embryology to study the growth and development of an embryo inside an egg. The term “candling” is understood herein to mean using a light source of sufficient strength directed at an egg enabling the detection of any structures inside an egg, preferably at minimum the air cell. A candling unit typically is a system comprising one or more light sources and one or more detectors.

Cleaning herein is understood to mean removing undesired substances, such as dirt, infectious agents, and other impurities, from an object or environment. Cleaning can be achieved by a variety of different means, for example by flushing with water or a solution containing a soap or detergent, using sound waves to shake particulates loose, using steam cleaning, applying one or more disinfectants, subjecting the object or environment to a sufficiently high temperature to kill or otherwise inactivate infectious agents or by thermal cleaning; a combined process involving pyrolysis and oxidation.

Characteristics of an egg are understood to mean either characteristics of the egg itself or characteristics of the embryo inside the egg. Examples of such characteristics are sex, feeding state, health status or developmental status. The feeding state is the amount and quality of beneficial nutrients inside the embryo or the egg or the ratio between both. The health status is the degree of the state of physical, anatomic, physiological well-being in which disease and infirmity are absent. The developmental status is the degree of development of the biological, biochemical and physical features of the embryo.

In the context of the present invention, the term solvent is a material capable of dissolving or dispersing or emulsifying another material.

Contacting the sample with other materials is meant to be understood as adding the sample to other materials, including other materials in solvents or emulsions, or vice versa. The adding can be done in tubes, reaction tubes, instruments for holding comprising one or more sample containers such as microplates, or other containers commonly used in a laboratory for handling or preparation of samples. During or after adding the sample or the other materials to each other they can be blended, mixed and/or incubated in order to optionally react the sample or an analyte therein and one or more of the other materials with each other.

Other materials are meant to be understood to be materials, in particular a fluid or a material in solution, used for further processing of the sample in order for one or more characteristics of the egg or the embryo to be assessed. Other materials comprise reference material, a diluent, a solvent, an enzyme, a binding agent or other material reacting with or binding to an analyte in the sample.

Reference material advantageously comprises a material, sufficiently homogeneous and stable with respect to one or more specified properties, which has been established to be fit for its intended use in a measurement process.

A conveyor is a commonly known piece of mechanical handling equipment that moves materials from one location to another. A conveyor system in the egg industry typically transports eggs from the hens throughout a facility, from any of to any of: collection, grading, incubating, hatching, sorting, packing or shipping. Eggs are commonly placed in trays or flats or can be placed directly on the conveyor. Conventional incubating or setting trays include the Chick Master® 54 tray, the Jamesway® 42 tray, and the Jamesway® 84 tray (in each case, the number indicates the number of eggs carried by the tray). There are some incubating trays, such as the La Nationale® incubating tray, which are sufficiently large enough to include an even higher total number of eggs, such as 132. Eggs can also be transported in a variety of other ways such as for example by a single carrier or a group of carriers comprising an egg accommodation and an egg clamping system as described in WO2019096372A1.

A tray herein is understood to mean a type of product created and designed in various colors, materials, mechanisms, shapes, sizes and styles used to hold and protect a specific number of eggs.

A detector is herein understood to mean a device or instrument designed to detect the presence of structures inside an egg or alternatively light or sound waves passing through or reflecting from the inside of the egg. The waves comprise light, other electromagnetic radiation or ultrasound sound waves. The detector comprises a sensor that detects and conveys information used to make an image. Two main types of electronic image sensors are the charge-coupled device (CCD) and the active-pixel sensor (CMOS sensor). Alternatively, a Quanta Image Sensor can be used.

The central egg axis is also commonly known as the major axis of the egg. The major axis spans the greatest possible distance between the tip of the pointed side of the egg and the base of the blunt side of the egg.

Fluid connection is understood herein as to be a connection between two or more systems comprising gas and/or fluid as a means of transporting substances between the two or more systems. This connection is considered to be enclosed and the gas and/or fluid is not in contact with the external environment, which can be achieved for example by using tubing.

Electrospray ionization (ESI) is a technique used in mass spectrometry to produce ions using an electrospray in which a high voltage is applied to a liquid to create an aerosol. It is especially useful in producing ions from macromolecules because it overcomes the propensity of these molecules to fragment when ionized. ESI is different from other ionization processes (e.g. matrix-assisted laser desorption/ionization (MALDI)) since it may produce multiple-charged ions, effectively extending the mass range of the analyser to accommodate the kDa-MDa orders of magnitude observed in proteins and their associated polypeptide fragments. ESI is a so-called ‘soft ionization’ technique, since there is very little fragmentation. This can be advantageous in the sense that the molecular ion (or more accurately a pseudo molecular ion) is always observed, however very little structural information can be gained from the simple mass spectrum obtained. Another important advantage of ESI is that solution-phase information can be retained into the gas-phase.

Matrix-assisted laser desorption/ionization (MALDI) is an ionization technique that uses a laser energy absorbing matrix to create ions from large molecules with minimal fragmentation. It is similar in character to electrospray ionization (ESI) in that both techniques are relatively soft (low fragmentation) ways of obtaining ions of large molecules in the gas phase, though MALDI typically produces far fewer multi-charged ions. MALDI methodology is a three-step process. First, the sample is mixed with a suitable matrix material and applied to a metal plate. Second, a pulsed laser irradiates the sample, triggering ablation and desorption of the sample and matrix material. Finally, the analyte molecules are ionized by being protonated or deprotonated in the hot plume of ablated gases, and then they can be accelerated into whichever mass spectrometer is used to analyze them.

Atmospheric-pressure chemical ionization (APCI) is an ionization method used in mass spectrometry which utilizes gas-phase ion-molecule reactions at atmospheric pressure (105 Pa), commonly coupled with high-performance liquid chromatography (HPLC). APCI is a soft ionization method similar to chemical ionization where primary ions are produced on a solvent spray. The main usage of APCI is for polar and relatively less polar thermally stable compounds with molecular weight less than 1500 Da.

Segmented flow or flow injection is understood to mean an approach to chemical analysis accomplished by injecting a plug of sample into a flowing carrier stream. The carrier solution and sample then meet at mixing points with reagents and react. The reaction product then flows through a detector. In addition, air can be optionally injected into the sample or reagent streams.

Egg herein is understood to mean a bird egg of a domesticated bird kept by humans for its eggs, meat or feathers. These birds are typically members of the superorder Galloanserae, but can also include for example ostriches, pigeons or doves. The eggs can be used to produce vaccines.

The present invention provides an automated egg determining system, comprising:
a. a conveyor system configured to transport one or more eggs to a sampling system;
b. a sampling system configured to extract a sample from one or more eggs;
c. a sample transfer system configured to receive the sample for transfer to an assaying system; and
d. an assaying system configured to receive the sample from the sample transfer system and to determine one or more characteristics of one or more eggs or the sample or an aliquot of a sample thereof.

Preferably, the system comprises a system to run an algorithm to exclude unfertilized eggs or eggs containing dead, un- or underdeveloped embryos.

Preferably, the characteristic is the sex of the embryo in the egg. Other preferred characteristics include the feeding state, health status or developmental status of the embryo in the egg.

Preferably, the automated egg determining system comprises a sorting system in communication with the assaying system and is configured to sort the egg based on the characteristics of the egg as determined by the assaying system. This enables a selection of certain eggs according to their determined characteristics.

Preferably, the sorting system comprises a holding area configured to hold and receive the egg for a predetermined period of time.

Preferably, the sorting system comprises an egg marking system, which comprises a means to mark an egg on the outside of the egg in line with the characteristic. Preferably, the sampling system comprises a candling unit which comprises one or more light sources and one or more detectors. This enables a visual or automatic determination of specific structures of the egg or the embryo inside the egg.

The light source, or light emission sources, may preferably comprise an incandescent or luminescent light source emitting electromagnetic waves, such as a halogen, gas-discharge, laser or light-emitting diode light source, in particular a high-intensity discharge, fluorescent, neon, argon, sulfur, metal-halide, plasma, xenon-flash, laser diode, chemical laser, gas laser, ion laser, solid-state laser light source. In a preferred embodiment, the light source is configured to emit light in a wavelength of from 300-2500 nm. According to some aspects, the light source may comprise a light emitting diode (LED) configured to emit light from the visible portion, or the infrared portion of the electromagnetic spectrum.

However, aspects of the present disclosure are not limited to the use of LEDs or infrared radiation. Various types of light sources may be utilized without limitation such as, for example, a laser diode source or a solid-state excitation source. The light source may emit light that is pulsed, time-sliced or modulated so as to avoid measurement errors caused by light emitted from adjacent light sources.

Preferably, the light source comprises an incandescent or luminescent light source. More preferably, the light source comprises a halogen, gas-discharge, laser or light-emitting diode light source. Preferred light sources comprise high-intensity discharge, fluorescent, neon, argon, sulfur, metal-halide, plasma, xenon-flash, laser diode, chemical laser, gas laser, ion laser, and/or solid-state laser light sources. Preferably, the light source is configured to emit light in a wavelength of from 300-2500 nm.

Preferably, the candling unit comprises a spacer system comprising a spacer object. This advantageous enables the positioning of the egg at a set distance.

Preferably, the system is configured to, and operable to position the egg to be in contact with the spacer object prior to, or during candling. Preferably, the spacer system comprises a light source. This reduces the scattering of incoming light to the egg and reflection of light from other surfaces. More preferably, the spacer object is of an essentially tubular shape, and located between the egg and the light source and comprises a lumen with two openings, one of which is opposite the egg, the other which is opposite the light source. This enables a channel through which light from the light source can reach the egg and thereby reduces the scattering of incoming light to the egg and reflection of light from other surfaces. Therefore, less light is needed to produce a better visualization of the egg and hence determination of specific structures of the egg or the embryo inside the egg. In addition, the reduced amount of light energy emitted towards the egg minimizes adverse effects of the light or the light energy on the egg or the embryo inside the egg.

Preferably, the spacer object has a spacing thickness in the range of from 0.01 to 20 mm. This short distance reduces the scattering of incoming light to the egg and reflection of light from other surfaces. More preferably, the spacer object is compressible at a compressive strength in the range of from 0 to 5 MPa, and optionally, exhibits material with an external shore hardness of from 0-90 Shore 000. The low compressive strength and shore hardness of the spacer object are important for minimizing any potential damage to the egg and specifically the egg shell when the egg is brought into contact with the spacer object.

Preferably, the spacer system comprises a spring configured to sustain the spacer in contact with the egg surface when compressed. This is important for minimizing any potential damage to the egg and specifically the egg shell when the egg is brought into contact with the spacer object.

More preferably, the spacer system comprises a bearing allowing rotation around a central axis. This allows (re)positioning of the egg for better access to specific structures of the egg or the embryo inside the egg during sampling of the egg.

Preferably, light originating from the light source present in the spacer system exits the spacer system via one opening. This enables the direction and focus of the light towards the egg. Preferably, the system is configured to, and operable to position the egg to be in lateral contact with one or more, preferably three or four, movable objects configured to move into or out of contact with the egg to respectively hold the egg or release the egg, preferably prior to, or during candling. This ensures that the egg is directed upwards aligning the central egg axis of the egg with the direction of gravity. When the egg is held into contact during candling a steady image of the egg can be captured by a detector. Release of the contact enables the egg to be (re)positioned. Advantageously, the movable objects comprise an elastic object that stores mechanical energy. Preferably, the elastic object that stores mechanical energy comprises a spring selected from the group consisting of a flat spring, a leaf spring or a coil spring.

Preferably, the sampling system comprises a means to determine the location of the air cell of the egg. Also, preferably, the sampling system comprises a means to determine the location of the allantois of the egg.

The detector or detector assembly comprises a plurality of detectors for receiving electromagnetic radiation.

The system may further include a detector assembly having a plurality of light sources and detectors for receiving electromagnetic radiation, such as light transmitted, or reflected by the egg during the candling operation.

In some instances, the detector assembly may be positioned opposite the light source assembly in axial alignment so as to form a plurality of light source/detector pairs capable of evaluating eggs in a high throughput manner. Each light source may contain light sources at one or more wavelengths so that the associated detector at each position may measure opacity at one or more wavelengths.

To measure several wavelengths with a single detector, light from the different light sources may be time-sliced or modulated to separate the opacity measurement at each wavelength. Preferably, the detector is positioned relative to the light source such that the detector can detect an image of the egg via light originating from the light source, however preferably not through the egg, but light source and detector are positioned at the same side. Advantageously, the light source is positioned between the egg and the detector. The detector may suitably be positioned at an angle of from 0-45° relative to a light ray originating from the light source.

According to a preferred embodiment of the invention, the detector may be a camera unit for capturing images of the illuminated upper portion of the egg, arranged such that the central egg revolution axis is substantially vertical, preferably by means of a light source arranged over the egg substantially on the revolution axis of the egg, and the acquisition of an image of the upper portion of the illuminated egg by a camera, arranged in the vicinity of the light source, preferably above the light source. The image acquisition axis of the camera may form an angle ranging between 0 and 70° with a reference plane that is perpendicular to the central axis of the egg.

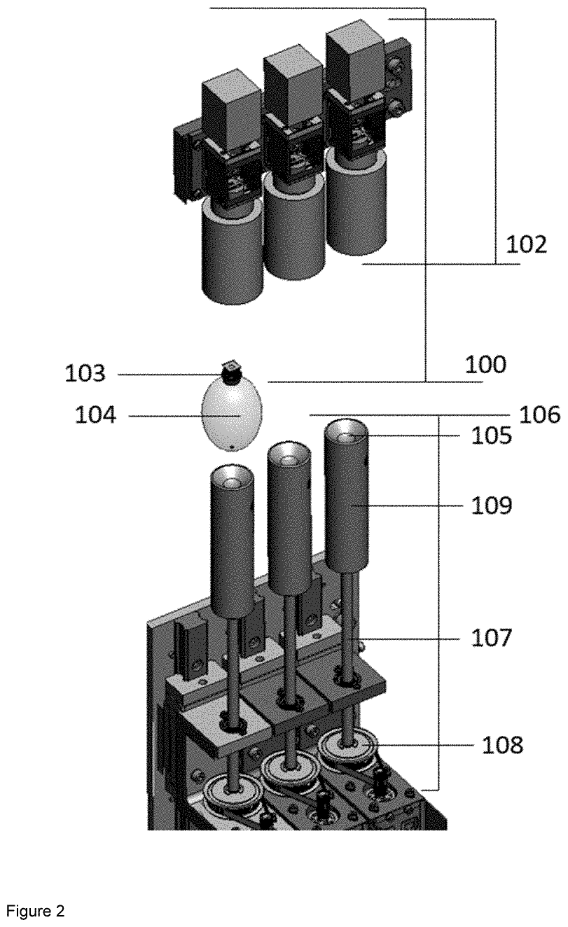

Preferably, at least one detector is positioned relative to the light source such that the detector can detect an image of the egg via light originating from the light source. Light from a light source can traverse the egg and/or reflect in any direction from the egg or the inside of the egg. More preferably, the light source is positioned between the egg and the detector. The position of the light source relative to the egg and the detector can be better understood by referring to FIGS. 2, 3 and 6. The light source can be located inside the spacer system which is positioned on top of the egg. Light emitted from the light source illuminates the egg and structures of the egg or the embryo inside the egg, which in turn can be detected by the detector located above the spacer system and light source. The light source is thus positioned at a point on a line between the centre of the egg and the detector. The detector is thus preferably located outside of the spacer system.

Preferably, the detector may be positioned at an angle of from 0-45° relative to a light ray originating from the light source. This enables the generation of a stronger signal by the detector. Preferably, the egg is positioned at a distance of from 0-30 mm from the light source of the candling unit for candling. This short distance reduces the scattering of incoming light to the egg and reflection of light from other surfaces. Therefore, less light is needed to produce a better visualization of the egg and hence determination of specific structures of the egg or the embryo inside the egg. In addition, the reduced amount of light energy emitted towards the egg minimizes adverse effects of the light or the light energy on the egg or the embryo inside the egg.

Preferably, the sampling system comprises a means to determine the location of a preferred extraction point of the egg. This enables an access location where one or more samples can be taken safely from one or more specific desired structures. The location of the extraction point herein refers to a position on the surface of the shell of the egg. This is the position where an opener will puncture the shell, creating an opening for a subsequent sampling needle to enter the interior of the egg, in order to take a sample, preferably of the allantoic fluid.

More preferably, the location of a preferred extraction point on the egg shell comprises a point on the shell of the egg on a line parallel to the central egg axis having a distance in the range of from 0.5 to 7 mm directly towards the centre of the egg from the lowest point of the air cell of the egg closest to the shell of the egg preferably, where the egg being positioned with its blunt side upwardly. This enables an access location to the air cell or any directly or indirectly adjacent structures.

Preferably, the sampling system comprises one or more openers to open a part of the egg shell of one or more eggs.

Preferably, the sampling system is configured to position the egg and the opener to bring the opener and/or the egg into contact with the opener at the preferred extraction point, or to position the egg and/or the opener in the trajectory of the opener movement towards the preferred extraction point.

More preferably, the sampling system is configured to position the egg and/or the opener, based on the central egg axis and the opener trajectory, at an angle of from 0-90°, or preferably 15-90°, with respect to the direction of the opener trajectory towards the preferred extraction point. This enables an ideal angle to minimize the amount of energy required to open the egg, thereby minimizing the risk of damaging the egg or any structures inside the egg. This is preferably done with the egg's blunt side facing upwards

Preferably, the sampling system comprises one or more extractors to extract a sample from one or more eggs. More preferably, the sampling system comprises one or more extractors to extract a sample from the allantois of one or more eggs.

Preferably, the sampling system is configured to position the egg and extractor relative to each other to bring the egg and extractor into contact at the preferred extraction point, or to position egg and/or extractor in a position in line with the trajectory of the extractor towards the preferred extraction point. More preferably, the sampling system is configured to position the egg and/or extractor, respectively, based on the central egg axis at an angle of from 0-90°, or preferably 0-45°, with respect to the extractor trajectory towards the preferred extraction point. This enables an ideal access and access trajectory of the extractor to specific desired structures of the egg for sampling.

Preferably, the extractor is configured to, and operable to traverse the air cell of the egg by a distance in the range of from 0.5 to 9 mm, preferably 3 mm, and to enter the allantois of the egg. Preferably, the extractor is configured to remove a volume of from 100 nl to 500 μl from the egg.

Preferably, the sampling system comprises a system to clean the extractor before or after extracting one or more samples from the egg. This is to reduce contamination of the eggs by pathogens and to reduce cross-contamination of samples from the eggs.

Advantageously, the sample transfer system comprises a liquid handling robot. Preferably, the sample transfer system is configured to hold and transfer the extracted sample via one or more instruments for holding comprising a multitude of sample containers. This enables the hold and transfer of the sample in, for example, a microtiter plate for further processing or analysis at a later stage.

Preferably, the sample transfer system is configured to contact the sample or an aliquot thereof with other materials. This to enable dilution and/or reaction of the sample with other reagents necessary to determine the amount and/or presence of one or more analytes for determination of one or more characteristics of the sample and by extension the egg or the embryo inside the egg.

Advantageously, the sample transfer system is configured to contact the sample or an aliquot thereof with a known amount of reference material. This to enable the determination the amount and/or presence of one or more analytes for determination of one or more characteristics of the sample and by extension the egg or the embryo inside the egg.

Preferably, the sample transfer system is configured to blend the sample or an aliquot thereof with other materials or a known amount of reference material.

Preferably, the sample transfer system or assaying system comprises segmented flow or flow injection. This enables an automated and segmented in-line transfer and/or reaction of samples prior to analysis.

Preferably, the assaying system comprises a system to detect molecules with a concentration of from 10⁻⁷ mol/m³-10⁻² mol/m³ in a sample.

Preferably, the assaying system is configured to generate a test result from an assay for detecting an analyte in a sample or an aliquot thereof, optionally contacted with other materials, within a time period of from 0.1 to 6 seconds after receiving the sample or an aliquot thereof from the sample transfer system. Preferably, the sample transfer system is configured to transfer a sample, or an aliquot thereof, optionally contacted with other materials, of a volume of from 1 to 1000 nl to the assaying system.

Preferably, the assaying system comprises one or more mass spectrometers, gas chromatographs, ion-mobility spectrometers, nuclear magnetic resonance spectrometers, Raman spectrometers, infrared spectrometers or electronic noses.

Preferably, the assaying system comprising a mass spectrometer further comprises electrospray ionization, matrix-assisted laser desorption/ionization or atmospheric-pressure chemical ionization. Advantageously, the electronic nose comprises one or more sensors which comprise one or more of the following types: a protein which binds specific molecules, a metal-oxide-semiconductor, a conducting polymer, a polymer composite, a quartz crystal microbalance or a surface acoustic wave.

Preferably, the sample transfer system comprises a sample aspiration tube and an injection valve, the injection valve being configured to alternatively apply a reduced pressure to a first fluid source and to a second fluid source, in each case via the sample aspiration tube, the first fluid source for filling a sample loop with samples, and the second fluid source for flushing the aspiration tube.

Preferably, the sample transfer system also comprises a system that is configured to transfer the extracted sample or an aliquot thereof, optionally contacted with other materials, and configured and adapted to perform a process comprising the following steps:
a. ejecting an aliquot from the sample by applying sound energy to an amount of the extracted sample;
b. entraining the ejected aliquot in a gas or liquid stream; and
c. transporting the entrained aliquot into the analyser using the gas or liquid stream.

Accordingly, the sample transfer system comprises an ejector using sound energy; and a gas or fluid conduit in communication with the ejector.

Preferably, the assaying system is configured to generate a test result from an assay for detecting an analyte in the sample or an aliquot thereof, optionally contacted with other materials, when used in the process comprising the following steps:
a. ionizing the analyte; and
b. detecting a number and/or amount of compounds in the analyte by a mass spectrometer, wherein the amount of analyte is related to the amount of analyte in the sample or an aliquot thereof, or a known amount of reference material in the sample or an aliquot thereof.

Accordingly, the assaying system preferably comprises an ionisation unit in communication with the gas or fluid conduit; and a mass spectrometer unit.

Advantageously, the sampling system is in a fluid connection with the sample transfer system. This enables direct in-line transfer of samples from the sampling system to the sample transfer system, thereby reducing the time required for transfer and any influence of external factors on the sample.

Preferably, the sample transfer system is in a fluid connection with the assaying system. This enables direct in-line transfer of samples from the sample transfer system to the assaying system, thereby reducing the time required for transfer and any influence of external factors on the sample.

Preferably, the sample transfer system is in a fluid connection with the sampling system and the assaying system. This enables direct in-line transfer of samples from the sampling system to the assaying system, thereby reducing the time required for transfer and any influence of external factors on the sample. Preferably, this comprises a two-way valve essentially without dead volume, and preferably a bubble detector, allowing to measure the passing aliquots and aligning them with a specific sample, and respectively, a particular egg.

The present invention also provides a method of determining an egg comprising a system to any of the systems described above.

The present invention also provides a method of determining eggs preferably involving the steps of:
a. candling one or more eggs;
b. automatically extracting one or more samples from one or more eggs;
c. transferring the sample to be analyzed;
d. automatically analyzing the sample or an aliquot of a sample thereof and determining one or more characteristics of the egg.

Preferably, the method of this invention comprises an additional step of positioning the egg in front of a light source and in contact with a spacing system preferably prior to, or during candling.

Preferably, the method of this invention comprises an additional step of determining the location of the air cell of the egg.

Preferably, the method of this invention comprises an additional step of determining the location of the allantois of the egg.

Preferably, the method of this invention comprises an additional step of determining the location of a preferred extraction point of the egg.

Preferably, the method of this invention comprises an additional step of holding the eggs while assigning the one or more characteristics, assigning the one or more characteristics, and optionally sorting the eggs according to the one or more characteristics.

The present invention additionally provides the use of a system for automated determination of a characteristic in a multitude of eggs according to any of the systems described above, and to multitudes of eggs that share a characteristic after being assigned and sorted.

### DETAILED DESCRIPTION OF THE FIGURES

The invention will now be discussed with reference to the figures, which show preferred exemplary embodiments of the subject invention.

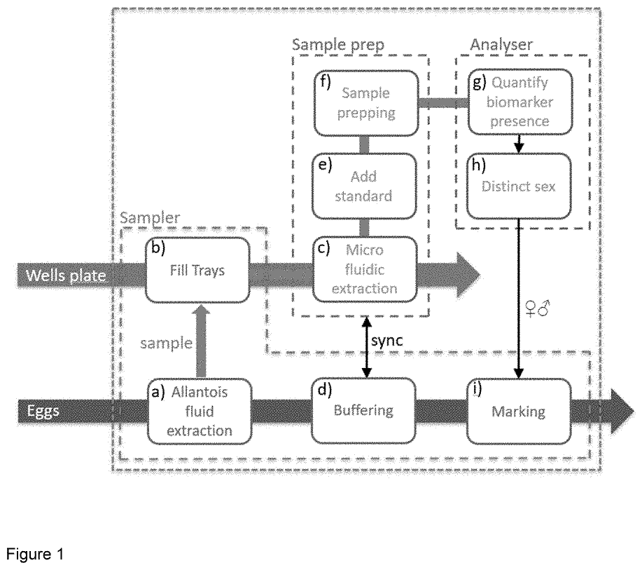

FIG. 1 shows a flow chart for a system for taking samples from eggs, transferring the samples to an assaying system and determining one or more characteristics of the eggs. A sample from the allantois (e.g. allantoic fluid) is taken from an egg at step a). The sample is transferred to a microtiter or wells plate in step b), which is used to collect a number of samples from one or more eggs. The samples are optionally contacted with a reference standard material or other materials and optionally blended in steps e) and f), while the egg is held in place in a buffering positioning in step d). An analyser runs an assay to determine the level of one or more biomarkers in step g) and reports the outcome of the assay for one or more characteristics (e.g. sex) in step h). The egg can then be marked externally for one or more characteristics in step i) for subsequent sorting.

FIG. 2 shows a model of a device capable of holding one or more eggs, which is also capable of rotating the egg and/or positioning the egg relative to a candling unit. An egg (104) is held in place by a vacuum or a mechanical means (105) of an egg manipulator (106). An extender (107) is configured to position the egg (104) towards a spacer object (103) and in front of a candling unit (100), comprising one or more detectors (102), one or more lighting sources (not shown) and in this model a spacer system (103). In this model the lighting source is located inside the spacer system (103). An egg rotator (108) is configured to rotate the egg manipulator (106) and by extension also the egg (104) and optionally a part of or the whole spacer system (103) when the egg (104) is positioned securely against the spacer system (103) and the spacer system (103) comprises a bearing.

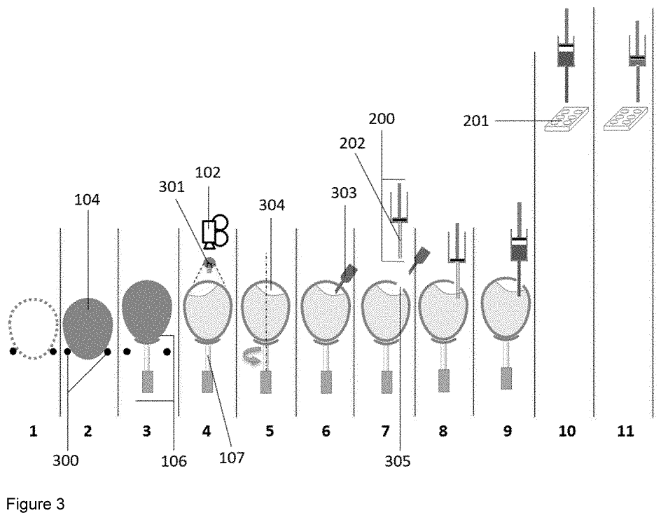

FIG. 3 shows a model for an apparatus containing several extractors, capable of moving the extractors from and to an instrument for holding comprising a multitude of sample containers. The apparatus contains one or more extractors (200) each comprising a hollow elongated object (202). This model can move the one or more extractors to and from a position above one or more eggs to and from an instrument for holding comprising a multitude of sample containers (e.g. a microtiter plate) (201).

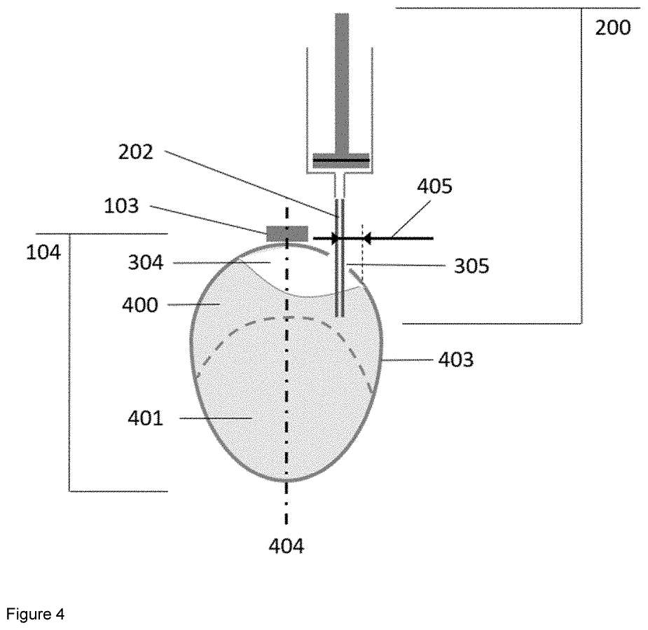

FIG. 4 shows a schematic overview of different steps of a system for taking one or more samples from one or more eggs. In step 1 an empty egg holder or tray (300) is present. In step 2 an egg (104) is placed blunt side up on the egg holder or tray (300). In step 3 an egg manipulator (106) is configured to hold the egg securely and is configured to lift the egg (104) from the egg holder or tray (300) via an extender (107). In step 4 the egg (104) is positioned by the egg manipulator (106) in front of a candling unit comprising a light source (301) and a detector (302). (Not shown) The top of the egg (104) is positioned by the extender (107) of the egg manipulator (106) against a spacer system and three or four lateral sides of the egg (104) each are positioned against respectively three or four flat springs. Following this the egg is candled. In step 5 a system which is configured to run an algorithm for determining the location of the air cell (304) of the egg (104) and a preferred extraction point (305) directs the egg manipulator (106) to rotate the egg (104) based on the information gathered by the detector (302). (Not shown) Prior to rotating the egg (104) the three or four lateral springs are moved out of contact with the egg (104). In step 6 the egg (104) and/or an opener (303) are positioned relative to each other in order for the opener (303) and subsequently the extractor (200) to contact the preferred extraction point (305). The opener (303) opens the egg (104). In step 7 the opener (303) is retracted from the egg (104) and the extractor (200) comprising a hollow elongated object (202) is positioned above the preferred extraction point (305). In step 8 the hollow elongated object (202) of the extractor (200) passes the preferred extraction point (305), enters the egg (104) and traverses the air cell (304). The distal end of the hollow elongated object (202) enters the allantois or a different structure of the egg (104). In step 9 the extractor (200) removes a sample from the egg (104). In step 10 the extractor is removed from the egg (104) towards an instrument for holding comprising a multitude of sample containers (e.g. a microtiter plate) (201). In step 11 the extractor (200) ejects the sample in the microtiter plate (201).

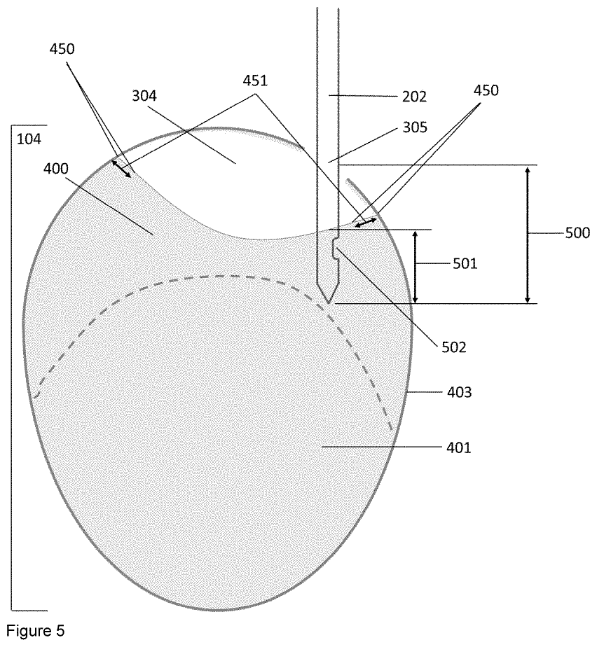

FIG. 5 shows a detailed schematic cross section of an extractor positioned in a (chicken) egg at the moment before taking a sample. The hollow elongated object (202) of an extractor (200) has entered an egg (104) through a preferred extraction point (305) and traversed the air cell (304) in order to remove a sample from the allantois (400) of the egg (104) without entering any underlying anatomical structures (401). The blunt end of the egg (104) is positioned against a spacer system (103). The location of the preferred extraction point (305) comprises a point on the shell (403) of the egg (104) on a line parallel to the central egg axis (404) having a distance of from 0.5-7 mm to an intersection (450) of the air cell (304) with the allantois (400) within a distance of from 0-2 mm (451) from the shell (403) of the egg (104), the distance to the intersection measured in the direction perpendicular to the central egg axis (404).

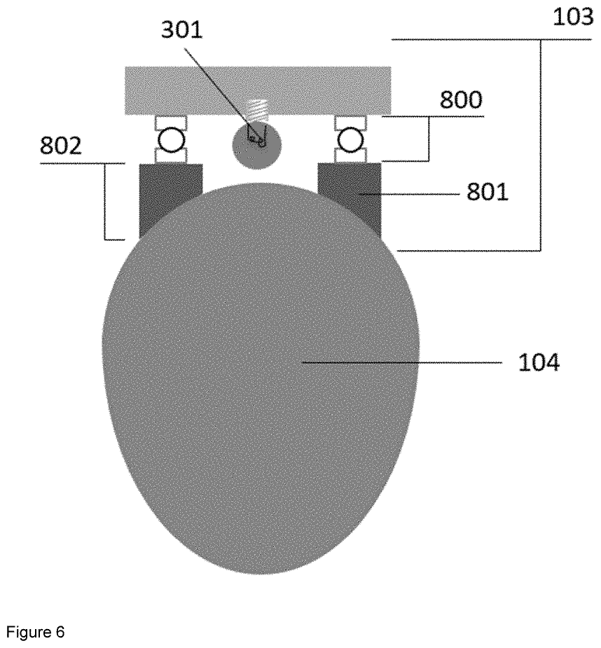

FIG. 6 shows a schematic cross section of a hollow elongated object of an extractor positioned in an egg. The hollow elongated object (202) of an extractor (200) has entered an egg (104) through a preferred extraction point (305) and traversed the air cell (304) in order to remove a sample from the allantois (400) of the egg (104) without entering any underlying anatomical structures (401). The distance of which the hollow elongated object (202) has traversed through the egg (104) from the preferred extraction point (305) is termed the needle depth (500). The distance of which the hollow elongated object (202) has traversed the allantois (400) is termed the penetration depth (501). The hollow elongated object (202) comprises a lateral opening (502) configured to remove a sample from the allantois (400).

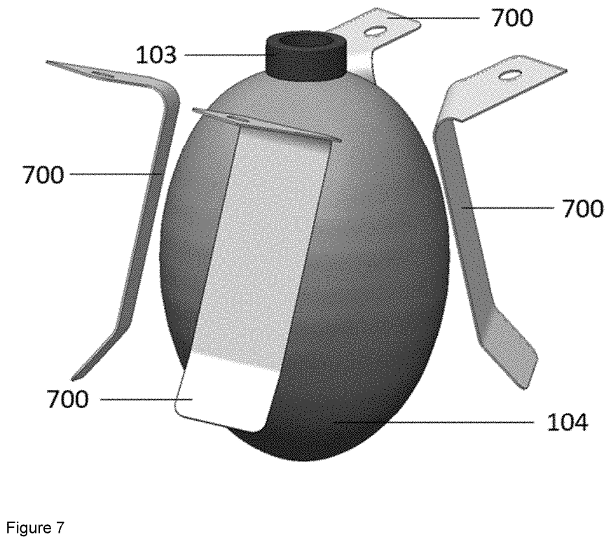

FIG. 7 shows a schematic overview of the egg and a spacer system during candling. The blunt side of an egg (104) is positioned against a spacer object (801) of the spacer system (103). The spacer system (103) comprises a light source (301) and a bearing (800). The spacer object has a thickness (802) and comprises an opening towards the egg (104) and an opening towards the light source (301). Light from the light source (301) exits the spacer system (103) only via the egg (104), thereby reducing reflection of the light from unwanted structures and enabling detection of structures within the egg by a detector (not shown).

FIG. 8 shows a schematic of the egg in contact with a spacer object and out of contact with flat springs prior to being rotated. The blunt side of an egg (104) is positioned against a spacer system (103). Immediately prior to rotating the egg (104) and after candling each of the lateral springs (700) are moved out of contact with the egg (104). (Not shown) Prior to and during candling the egg is positioned to be in contact with all lateral springs (700) enabling the positioning of the central egg axis parallel to the direction of gravity.

The present system permits an automated analysis of a multitude of eggs. Based on a prototype embodiment, a speed of processing is considered feasible in the range of from several hundreds to several thousands of eggs per hour, with an unprecedented selection exactness for the desired characteristic, in particular sex of the embryo in the egg, developmental stage, health and/or other characteristics.

The present invention has been described above with reference to a number of exemplary embodiments as shown in the drawings. Modifications and alternative implementations of some parts or elements are possible and are included in the scope of protection as defined in the appended claims.

1. An automated egg determining system, comprising:
a. a conveyor system configured to transport one or more eggs to a sampling system;
b. a sampling system configured to extract a sample from one or more eggs, the sampling system comprising a candling unit comprising one or more light sources and one or more detectors, wherein each of the at least one detector is positioned relative to the light source such that the detector can detect an image of the egg via light originating from the light source,
c. a sample transfer system configured to receive the sample for transfer to an assaying system; and
d. an assaying system configured to receive the sample from the sample transfer system and to determine one or more characteristics of one or more eggs or the sample or an aliquot of a sample thereof.

2. The system according to claim 1, comprising a sorting system in communication with the assaying system and is configured to sort the egg based on the characteristics of the egg as determined by the assaying system.

3. The system according to any one of claims 1 to 2, wherein the detector is positioned at an angle of from 0-45° relative to a light ray originating from the light source.

4. The system according to any one of claims 1 to 3, wherein the egg is positioned at a distance of from 0-30 mm from the light source of the candling unit for candling.

5. The system according to any one of claims 1 to 4, wherein the candling unit comprises a spacer system comprising a spacer object.

6. The system according to claim 5, wherein the spacer system comprises a light source, preferably wherein the light source is positioned inside the spacer object.

7. The system according to any one of claim 5 or 6, wherein the spacer object is of an essentially tubular or torus shape, and is located between the egg and the light source, and comprises a lumen with two openings, one of which positioned opposite the egg, the other opposite the light source.

8. The system according to any one of claims 5 to 7, wherein the spacer object has a spacing thickness in the range of from 0.01 to 20 mm.

9. The system according to any one of claims 5 to 8, wherein the spacer object is compressible at a compressive strength in the range of from 0 to 5 MPa, and optionally, exhibits material with an external shore hardness of from 0-90 Shore OOO.

10. The system according to any one of claims 5 to 9, wherein the spacer system comprises a spring configured to sustain the spacer in contact with the egg surface when compressed.

11. The system according to any one of claims 5 to 10, wherein the spacer system comprises a bearing allowing rotation around a central axis.

12. The system according to any one of claims 5 to 11, wherein light originating from the light source in the spacer system exits the spacer system via one opening, to illuminate the egg when in contact with the egg.

13. The system according to any one of claims 5 to 12, configured to, and operable to position the egg to be in contact with the spacer object prior to, or during candling.

14. The system according to any one of claims 1 to 13, configured to, and operable to position the egg to be in lateral contact with one or more, preferably three or four, movable objects configured to move into or out of contact with the egg to respectively hold the egg or release the egg, preferably prior to, or during candling.

15. The system according to claim 14, wherein the movable objects comprise an elastic object that stores mechanical energy.

16. The system according to claim 15, wherein the elastic object that stores mechanical energy comprises a spring selected from the group consisting of a flat spring, a leaf spring, a trapezoidal or square spring or a coil spring.

17. The system according to any one of claims claims 1 to 16, wherein the light source comprises an incandescent or luminescent light source, preferably emitting electromagnetic radiation.

18. The system according to any one of claims 1 to 17, wherein the light source comprises a halogen light source, a gas-discharge light source, a laser light source or light-emitting diode light source.

19. The system according to any one of claims 1 to 18, wherein the light source comprises a high-intensity discharge light source, fluorescent light source, neon light source, argon light source, sulfur light source, metal-halide light source, plasma light source, xenon-flash light source, laser diode, chemical laser, gas laser, ion laser, or a solid-state laser light source.

20. The system according to any one of claims 1 to 19, wherein the light source is configured to emit light in a wavelength in the range of from 300-2500 nm.

21. The system according to any one of claims 1 to 20, wherein the sampling system comprises a detector unit to determine the location of the air cell of the egg.

22. The system according to any one of claims 1 to 21, wherein the sampling system comprises a detector unit to determine the location of the allantois of the egg.

23. The system according to any one of claims 1 to 22, wherein the sampling system comprises a detector unit to determine the location of a preferred extraction point of the egg.

24. The system according to claim 23, wherein the location of a preferred extraction point on the egg shell comprises a point on the shell (403) of the egg (104) on a line parallel to the central egg axis (404) having a distance of from 0.5-7 mm to an intersection (450) of the air cell (304) with the allantois (400) within a distance of from 0-2 mm (451) from the shell (403) of the egg (104), the distance to the intersection measured in the direction perpendicular to the central egg axis (404).

25. The system according to any one of claims 1 to 24, wherein the sampling system comprises one or more openers to open a part of the egg shell of the one or more eggs.

26. The system according to any one of claims 1 to 25, wherein the sampling system comprises one or more extractors to extract a sample from the one or more eggs.

27. The system according to claim 26, wherein the sampling system comprises one or more extractors to extract a sample from the allantois of one or more eggs.

28. The system according to claim 25, wherein the sampling system is configured to position the egg and the opener to bring the opener and/or the egg into contact with the opener at the preferred extraction point, or to position the egg and/or the opener in the trajectory of the opener movement towards the preferred extraction point.

29. The system according to any one of claim 25 or 28, wherein the sampling system is configured to position the egg and/or the opener, based on the central egg axis and the opener trajectory, at an angle of from Oy to 90° with respect to the direction of the opener trajectory towards the preferred extraction point.

30. The system according to any one of claim 26 or 27, wherein the sampling system is configured to position the egg and extractor relative to each other to bring the egg and extractor into contact at the preferred extraction point, or to position egg and/or extractor in a position in line with the trajectory of the extractor towards the preferred extraction point.

31. The system according to any one of claim 26, 27 or 30, wherein sampling system is configured to position the egg and/or extractor, respectively, based on the central egg axis at an angle of from 0 to 90° with respect to the extractor trajectory towards the preferred extraction point.

32. The system according to any one of claim 26, 27, 30 or 31, wherein the extractor is configured to, and operable to traverse the air cell of the egg by a distance in the range of from 0.5 to 9 mm, preferably 3 mm, and to enter the allantois of the egg.

33. The system according to any one of claims 26, 27, 30 to 32, wherein the extractor is configured to extract a sample with a volume of from 100 nl to 500 μl from the egg.

34. The system according to any one of claims 26, 27, 30 to 33, wherein the sampling system comprises a system to clean the extractor before and/or after extracting one or more samples from the egg.

35. The system according to any one of claims 1 to 34, wherein the sample transfer system is configured to hold and transfer the extracted sample via one or more instruments for holding a multitude of sample containers.

36. The system according to any one of claims 1 to 35, wherein the sample transfer system is configured to contact the sample or an aliquot thereof with other materials.

37. The system according to any one of claims 1 to 36, wherein the sample transfer system is configured to contact the sample or an aliquot thereof with a known amount of reference material.

38. The system according to claim 36 or 37, wherein the sample transfer system is configured to blend the sample or an aliquot thereof with other materials or a known amount of a reference material.

39. The system according to any one of claims 1 to 38, wherein the sample transfer system or assaying system utilizes segmented flow or flow injection.

40. The system according to any one of claims 1 to 39, wherein the assaying system comprises a system to detect molecules with a concentration of from 10⁻⁷ mol/m³-10⁻² mol/m³ in a sample.

41. The system according to any one of claims 1 to 40, wherein the assaying system is configured to generate a test result from an assay for detecting an analyte in a sample or an aliquot thereof, optionally contacted with other materials, within a time period of from 0.1 to 6 seconds after receiving the sample or an aliquot thereof from the sample transfer system.

42. The system according to any one of claims 1 to 41, wherein the sample transfer system is configured to transfer a sample, or an aliquot thereof, optionally contacted with other materials, of a volume of from 1 to 1000 nl, to the assaying system.

43. The system according to any one of claims 1 to 42, wherein the assaying system comprises one or more mass spectrometers, gas chromatographs, ion-mobility spectrometers, nuclear magnetic resonance spectrometers, Raman spectrometers, infrared spectrometers or electronic noses.

44. The system according to any one of claims 1 to 43, wherein the assaying system comprising a mass spectrometer further comprises electrospray ionization, matrix-assisted laser desorption/ionization or atmospheric-pressure chemical ionization.

45. The system according to any one of claims 1 to 44, wherein the electronic nose comprises one or more sensors which comprise one or more of the following types; a protein which binds specific molecules, a metal-oxide-semiconductor, a conducting polymer, a polymer composite, a quartz crystal microbalance or a surface acoustic wave.

46. The system according to any one of claims 1 to 45, wherein the sample transfer system comprises a sample aspiration tube and an injection valve, the injection valve being configured to alternatively apply a reduced pressure to a first fluid source and to a second fluid source, in each case via the sample aspiration tube, the first fluid source for filling a sample loop with samples, and the second fluid source for flushing the aspiration tube.

47. The system according to any one of claims 1 to 46, wherein the sample transfer system comprises a system that is configured to transfer the extracted sample or an aliquot thereof, optionally contacted with other materials, according to the following steps:
a. an aliquot from the sample is ejected by applying sound energy to an amount of the extracted sample;
b. the ejected aliquot is entrained in a gas or liquid stream; and
c. the entrained aliquot is transported into the analyser using the gas or liquid stream.

48. The system according to any one of claims 1 to 47, wherein the assaying system is configured to generate a test result from an assay for detecting an analyte in the sample or an aliquot thereof, optionally contacted with other materials, comprising the following steps:
a. the analyte is ionized; and
b. the amount of analyte is detected by a mass spectrometer, wherein the amount of analyte is related to the amount of analyte in the sample or an aliquot thereof or a known amount of reference material in the sample or an aliquot thereof.

49. The system according to any one of claims 1 to 48, wherein the sample transfer system comprises a liquid handling robot.

50. The system according to any one of claims 1 to 49, wherein the sampling system is in a fluid connection with the sample transfer system.

51. The system according to any one of claims 1 to 50, wherein the sample transfer system is in a fluid connection with the assaying system.

52. The system according to any one of claims 1 to 51, wherein the sample transfer system is in a fluid connection with the sampling system and the assaying system.

53. The system according to any one of claims 1 to 52, which comprises a system to run an algorithm to exclude unfertilized eggs or eggs containing dead, undeveloped or underdeveloped embryos.

54. The system according to any one of claims 1 to 53, wherein the characteristic is the sex of the embryo in the egg.

55. The system according to any one of claims 1 to 54, wherein the characteristic is the feeding state, health status or developmental status of the embryo in the egg.

56. The system according to any one of claims 2 to 55, wherein the sorting system comprises a holding area configured to hold and receive the egg for a predetermined period of time.

57. The system according to any one of claims 2 to 56, wherein the sorting system comprises an egg marking system, which comprises a means to mark an egg on the outside of the egg in line with the characteristic.

58. A method of determining a characteristic of an egg, the method comprising using a system according to any of claims 1 to 57.

59. A method for determining a characteristic of one or more eggs, comprising the steps of:
a. candling one or more eggs using a candling unit comprising one or more light sources and one or more detectors, wherein each of the at least one detector is positioned relative to the light source such that the detector can detect an image of the egg via light originating from the light source;
b. automatically extracting one or more samples from the one or more eggs;
c. transferring the one or more samples to an analyzing unit;
d. automatically analyzing the one or more samples or an aliquot thereof; and
e. determining one or more characteristics of the one or more eggs.

60. The method according to claim 59, comprising positioning the egg in contact with, preferably in front of, a light source and in contact with a spacing system, preferably prior to, or during candling.

61. The method according to any one of claims 59 to 60, comprising determining the location of the air cell of the egg, and/or the location of the allantois of the egg.

62. The method according to any one of claims 59 to 61, comprising holding the eggs while assigning the one or more characteristics, assigning the one or more characteristics, and optionally sorting the eggs according to the one or more characteristics, to obtain a multitude of eggs sharing a characteristic.

63. The method according to any one of claims 59 to 62, comprising determining the location of a preferred extraction point of the egg.

64. Use of a system according to any one of claims 1 to 57 for automated determination of a characteristic in a multitude of eggs.

65. A multitude of eggs sharing a characteristic, obtainable by the process according to any one of claims 61 to 64.
